(19) United States
(12) Patent Application Publication
(10) Pub. No.: US 2021/0317919 A1
(43) Pub. Date: Oct. 14, 2021

(54) FLUID MODULE, FLUID SYSTEM AND METHOD OF CLEANING A FLUID MODULE

(71) Applicant: Buerkert Werke GmbH & Co. KG, Ingelfingen (DE)

(72) Inventors: Alexander EQUIT, Ingelfingen (DE); Ralf KETTEMANN, Ingelfingen (DE)

(21) Appl. No.: 17/226,237

(22) Filed: Apr. 9, 2021

(30) Foreign Application Priority Data
Apr. 14, 2020 (DE) 10 2020 110 209.7

(51) Int. Cl. F16K 11/02; F16K 7/12; F16K 27/00

(52) U.S. Cl. CPC F16K 11/022; F16K 27/003; F16K 7/126

(57) ABSTRACT

A fluid module having a valve node is specified, which includes a base body delimited by an outer surface and having a central fluid space, and at least four fluid channels which are fluidically separated from each other and each open into the central fluid space, wherein at least two valve connections are provided on the outer surface, and wherein a coupling point is provided on the valve node, to which a coupling module is fastened, in which a connection channel is formed, which is fluidically connected to one of the fluid channels in the valve node, and wherein the coupling module includes a coupling valve which selectively unblocks and closes the connection channel. A fluid system having at least two fluid modules, and a method of cleaning a fluid system or a fluid module are furthermore specified.

### FIELD OF THE INVENTION

The invention relates to a fluid module having a valve node, to a fluid system having at least two fluid modules, and to a method of cleaning a fluid module or a fluid system.

### BACKGROUND

Fluid modules or fluid systems are used, among other things, for coupling and uncoupling processes in hygienic applications. One possible application example is the supply of cell cultures in a container with a supply medium, for example a nutrient solution, or the removal of a product grown by means of the cell cultures from the container. Such a process can take several weeks, wherein the container containing the cell cultures must be repeatedly coupled to a fluid system to supply the cell cultures.

During the multiple coupling or uncoupling of the containers, contaminants may occur in the fluid modules, which may lead to faulty batches if the contaminants reach the cell cultures or mix with the finished product. In particular, media transferred after coupling or uncoupling can be contaminated. This risk of occurring contamination is particularly high especially when a plurality of different containers are alternately supplied with different supply media by means of the fluid modules. A small amount of a first supply medium can remain in the fluid module and mix with a different supply medium subsequently flowing through the fluid module, which can render the cell cultures unusable.

Furthermore, contamination may also occur in the media-carrying area of the fluid module during assembly of the fluid module.

It is therefore an object of the invention to provide a fluid module or a fluid system for coupling and uncoupling processes which can be easily cleaned after each media transfer.

### SUMMARY

The invention provides a fluid module having a valve node which includes a base body delimited by an outer surface and having a central fluid space and at least four fluid channels which are fluidically separated from each other and each open into the central fluid space, wherein at least two valve connections are provided on the outer surface, and wherein a coupling point is provided on the valve node, to which a coupling module is fastened. A connection channel is formed in the coupling module, which is fluidically connected to one of the fluid channels in the valve node. The coupling module further comprises a coupling valve which selectively unblocks and closes the connection channel.

A valve node is understood to be a body having a plurality of inflows and a plurality of outflows for liquids or gases, all of the inflows and outflows being in fluid communication with each other via a central fluid space.

The fluid module according to the invention has the advantage that possibly contaminated areas can be cleaned in a defined manner after each coupling or uncoupling process before media are transferred through these areas again. The transferred media can be, in particular, a supply medium for cell cultures or a generated product.

In particular, the media-contacted areas in the base body of the valve node can be cleaned, so that the risk of contaminating media that are transferred after a coupling or uncoupling of the fluid module is minimized or excluded.

In addition, there is no need to clean the components of the fluid module prior to assembly of the fluid module. Thus, a complex sterilization of the components of the fluid module before assembly can be omitted.

The particularly good cleaning option results in particular from the fact that different flow paths can be formed through the valve node by means of the four fluid channels fluidically separated from each other, one of the flow paths serving to guide a supply media flow, one of the flow paths serving to guide a product flow, and a further flow path serving to guide a cleaning media flow. As all fluid channels open into the central fluid space, the latter can be cleaned by the cleaning media flow before a supply media flow or a product flow passes through the central fluid space.

Ultrapure steam, water for injection purposes or a chemical cleaning agent, for example, serves as a cleaning medium.

By means of the coupling module, in particular by means of the connection channel, media can be fed to a container particularly easily. For this purpose, a container or a line leading to the container can be connected to the coupling module, in particular fluidically connected to the connection channel. The container or line should be connected while the connection channel is closed by the coupling valve.

The fluid channels formed in the base body are, in particular, a supply media feeding channel, a cleaning media feeding channel, a transfer channel and an outlet channel. The transfer channel can be fluidically connected to the connection channel in the coupling module.

For cleaning purposes, it is possible to close the connection channel and thus also the transfer channel by means of the coupling valve as well as another of the fluid channels formed in the base body, in particular the supply media feeding channel, so that the central fluid space can be purged with a cleaning medium.

For example, one of the fluid channels formed in the base body may open out at the coupling point and is fluidically connected to the connection channel formed in the coupling module.

According to one example, the coupling module is non-destructively detachably fastened to the valve node. In this way, differently designed coupling modules can be selectively fastened to the valve node if required.

The coupling module can include a coupling valve housing having a valve seat, a closure body cooperating with the valve seat, and a valve actuator for the closure body. This makes it particularly easy to close or unblock the connection channel.

The valve seat formed in the coupling valve housing may be formed in particular in the connection channel and divides it into two sections.

A seal can be arranged at the coupling point between the valve node and the coupling module, which surrounds a discharge opening of the fluid channel opening into the coupling point. This ensures that a reliable sealing is realized in a simple manner when the coupling module is attached and that no fluid escapes at the interface.

The coupling point may be formed by a coupling flange formed by a projection of the base body, in particular wherein the base body is L-shaped and the coupling point is formed on an inner side of one of the legs of the L. In this way, the fastening of the coupling module to the base body of the valve node is simplified. In particular, the coupling module can be placed on the coupling flange and then fastened thereto.

The coupling point may have, for example, a coupling face against which the coupling module rests, in particular the coupling valve body, the coupling face extending at least in sections in an inclined manner relative to a flow direction of a fluid in the area of the coupling point. In other words, the inner sides of the legs of the L extend at least in sections at an obtuse angle to each other. This makes it particularly easy to mount the coupling module both from the top and from the front. This is particularly advantageous if the coupling module has to be replaced later.

The coupling face can run in an inclined manner in the area where the seal is arranged. In this way, the inclination prevents the seal from being damaged during assembly of the coupling module.

According to a further example, the base body is substantially parallelepiped-shaped, and the coupling point is provided on a side face of the parallelepiped. The fluid module thus has a particularly compact design.

A valve actuator may be provided at each valve connection of the base body. In this way, all fluid channels formed in the base body can be selectively closed or unblocked. More precisely, different flow paths can be switched through the valve node by respectively closing two fluid channels and opening two further fluid channels.

Positioning means and/or fastening means can be provided on the valve node and/or on the coupling module, in particular on the coupling valve housing, in order to fasten the coupling module in a defined position on the valve node. The positioning means ensure that the coupling module is arranged in a defined position on the valve node. Furthermore, the positioning means may already serve to hold the coupling module in a designated position until the coupling module is properly fastened by means of the fastening means.

The central fluid space may be openly accessible from at least one side of the base body and, in a plan view of said side, a respective web may be formed in the base body on both sides of the central fluid space, each of the two webs forming a valve seat and delimiting the central fluid space with respect to a respective one of the fluid channels adjacent thereto. The accessibility of the central fluid space has the advantage, on the one hand, that the central fluid space can be manufactured in a simple manner in the base body of the valve node. For example, the base body can be manufactured in one piece as a casting or milled part. By forming a web on both sides of the central fluid space in a plan view of one side, each of which forms a valve seat, it is possible to use a so-called multiport valve actuator, as a result of which the fluid module can be particularly compact. In particular, two valve seats can be operated by means of a single valve actuator.

A common closure body can be assigned to the two webs, the closure body being a diaphragm which can be selectively pressed against each of the webs in a sealing manner by a valve actuator. This also contributes to a compact design of the fluid module. In addition, the number of components can be kept low, which also has a positive effect on the manufacturing costs.

The invention further suggests a fluid system having at least two fluid modules which are designed as described above, wherein the individual fluid modules are connected to each other such that an output fluid channel of the fluid channels of one fluid module is fluidically connected to an input fluid channel of the fluid channels of the at least one further fluid module. In this way, any number of fluid modules can be lined up.

By means of such a fluid system, a plurality of containers can be supplied with a supply medium at the same time or a product flow can be discharged from several containers at the same time. The fluid system is thus particularly suitable for use in combination with so-called tub basin stacks, which have a plurality of separate receiving spaces in which cell cultures can be received.

By stringing together a plurality of fluid modules, the product flows discharged from the individual containers can be combined into a total product flow, which in turn can be collected into a product container.

The individual fluid modules of the fluid system can be operated independently of each other in parallel. This means that the fluid system can also be used to supply a plurality of containers in parallel with different supply media without the media mixing with each other.

The outlet channel of one fluid module can respectively be connected to the cleaning media feeding channel of another fluid module. This allows a cleaning media flow to be directed through all fluid modules of a fluid system to respectively clean the central fluid space of each fluid module. After flowing through the fluid system, the cleaning media flow can be collected in a cleaning container.

Both the fluid module and the fluid system may have a plurality of fluid modules are configured to be self-draining in at least one orientation in space. For example, the fluid module or the fluid system is self-draining in an installed position, in that there is a continuous slope from the fluid input to the fluid output. Self-draining is to be understood such that that no medium remains in the fluid system when the fluid flow stops, or even that no backup of the medium is produced.

Furthermore, the fluid module or the fluid system can be free of sinks in which a fluid can become trapped. In other words, the fluid system is free of dead spaces.

The invention also suggests a method of cleaning a fluid module or a fluid system each configured as described above, comprising the following steps:
closing two of the four fluid channels of a fluid module and unblocking the remaining fluid channels,
supplying a cleaning medium through one of the two unblocked fluid channels, wherein the central fluid space is flowed through, and
discharging the cleaning medium through the further unblocked fluid channel.

Due to the fact that the cleaning medium flows through and thus cleans the central fluid space, into which all fluid channels open, no contamination of the respective media flow occurs during a subsequent supply process or discharge of a product.

During cleaning, the cleaning medium can flow through the fluid system at a defined flow rate.

During cleaning, at least one of the two unblocked fluid channels can be repeatedly closed and reopened in a pulse-like manner. In other words, at least one valve is switched in a clocked manner. This causes a recoil in the fluid, in particular in the cleaning medium, and improves the cleaning. The effect created in this way is similar to the effect of sound waves. In this way, a sufficiently high flow energy is achieved in the space to be cleaned. Even process-critical areas such as stub lines can thus be cleaned in a reliable way.

In a fluid system, a valve arranged in the area of a drain may be closed in a pulse-like manner to generate the recoil, in particular the last valve in the row.

### BRIEF DESCRIPTION OF THE DRAWINGS

FIG. 1 schematically shows a fluid system according to the invention having a plurality of fluid modules according to the invention,

FIG. 2 shows a fluid system according to the invention having a plurality of fluid modules according to the invention, the fluid modules being each illustrated in different stages of assembly,

FIG. 3 shows a side view of the fluid system from FIG. 2,

FIG. 4 shows a sectional view of the fluid system from FIGS. 2 and 3,

FIG. 5 shows a fluid module according to the invention,

FIG. 6 shows an exploded view of a valve node having a coupling module,

FIG. 7 shows a sectional view through a part of the valve node and the coupling module,

FIG. 8 shows a base body of a valve node,

FIG. 9 shows a perspective sectional view of a part of the fluid module,

FIG. 10 shows a base body of a valve node,

FIG. 11 shows a perspective sectional view of a part of the fluid module,

FIG. 12 shows an exploded view of a part of a fluid module according to the invention in a further embodiment, and

FIG. 13 shows a perspective sectional view of the fluid module from FIG. 12.

### DETAILED DESCRIPTION

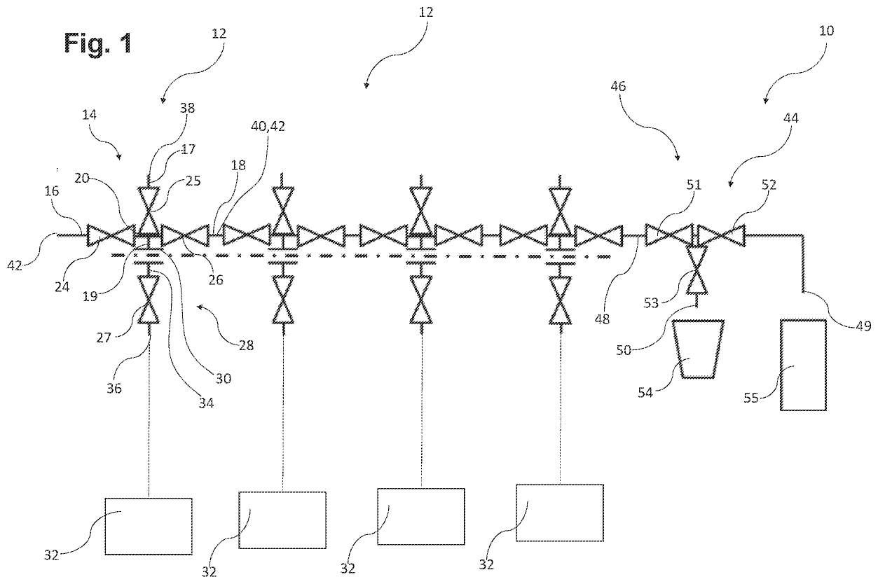

FIG. 1 schematically shows a fluid system 10 according to the invention having a plurality of fluid modules 12 according to the invention. The individual fluid modules 12 are fluidically connected to each other.

Each fluid module 12 comprises a valve node 14 having four fluid channels 16, 17, 18, 19 which are fluidically separated from each other and open into a central fluid space 20.

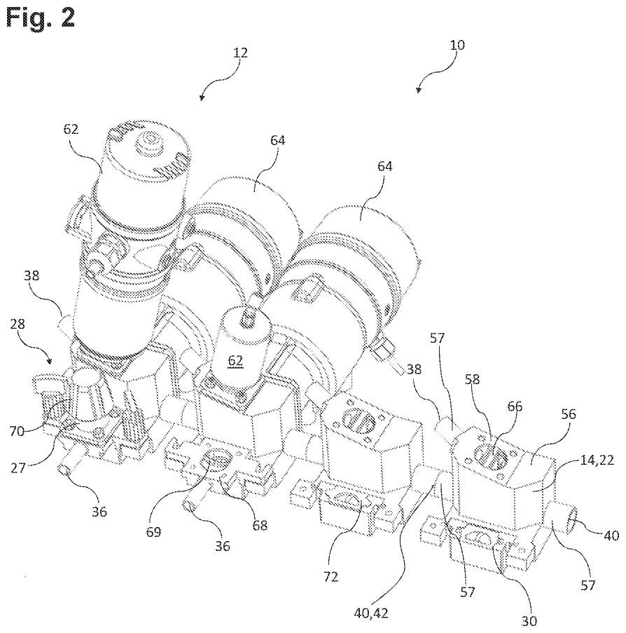

The fluid channels 16, 17, 18, 19 and the central fluid space 20 are formed in a base body 22 which can be seen in FIG. 2.

The fluid module 12 further comprises four individual valves 24, 25, 26, 27, each of which serves to block or unblock a fluid flow through an associated fluid channel 16, 17, 18, 19.

One of the individual valves 24, 25, 26, 27 is part of a coupling module 28, which is fastened to the valve node 14 at a coupling point 30. The corresponding individual valve 27 thus forms a coupling valve. The separation between the valve node 14 and the coupling modules 28 is illustrated by a dashed line in FIG. 1. The coupling modules 28 are shown slightly spaced from the valve nodes 14. In the assembled state, however, the coupling modules 28 are in direct contact with the valve nodes 14, in particular the coupling point 30.

The individual fluid modules 12 serve to supply a supply medium to, or remove a product from a container 32 connected to a fluid module 12. The container 32 is, for example, a so-called tub basin stack having a plurality of receiving spaces which separated from each other.

For example, each fluid module 12 can supply a different receiving space of the container 32. In this case, the containers 32 shown separately in FIG. 1 illustrate individual receiving spaces of a common tub basin stack.

The fluid channels 16, 17, 18, 19 formed in the valve node 14 are a cleaning media feeding channel (fluid channel 16), a supply media feeding channel (fluid channel 17), an outlet channel (fluid channel 18), and a transfer channel (fluid channel 19).

The transfer channel 19 opens into the coupling point 30 and is fluidically connected to a connection channel 34 formed in the coupling module 28.

The connection channel 34 has a connection 36 at its end facing away from the valve node 14, which is suitable for connecting a flexible fluid line to connect the fluid module 12 to the container 32.

Accordingly, each of the fluid modules 12 has a supply media input 38, a media output 40, a cleaning media input 42, and the connection 36.

In the fluid system 10 shown schematically in FIG. 1, the individual fluid modules 12 are connected to each other in that an output fluid channel of the fluid channels 16, 17, 18, 19 of one fluid module 12, more specifically the outlet channel 18, is fluidically connected to an input fluid channel of the fluid channels 16, 17, 18, 19 of an adjacent fluid module 12, more specifically the cleaning media feeding channel 16.

In particular, the media output 40 of a fluid module 12 is respectively connected to the cleaning media input 42 of a subsequent fluid module 12.

The valves 24, 26 assigned to the cleaning media feeding channel 16 and the outlet channel 18 serve as intermediate bulkhead valves, that is the individual fluid modules 12 can be fluidically separated from each other by means of the valves 24, 26.

By opening or closing the valves 24, 25, 26, 27 in a suitable manner, different media flows can be directed through the fluid system 10. For this purpose, two fluid channels of a fluid module 12 are selectively closed and the two other fluid channels of a fluid module 12 are opened.

More precisely, three different states of the fluid system 10 are possible.

In a first state (supply state), the containers 32 are supplied with a supply medium. For this purpose, the valves 25, 27 assigned to the supply media feeding channel 17 and the transfer channel 19 are open and the remaining valves 24, 26 are closed so that a supply media flow can flow from the supply media input 38 to the connection 36.

In a further state (removal state) of the fluid system 10, a product can be discharged from the containers 32. For this purpose, the valves 24, 25 assigned to the cleaning media feeding channel 16 and the supply media feeding channel 17 are closed, while the remaining valves 26, 27 are open. In this condition, a product can flow from the containers 32 to the media output 40.

If a plurality of fluid modules 12 are connected in series, as shown in FIG. 1, only the valve 24 closes the cleaning media feeding channel 16 in the first fluid module 12 as viewed in the direction of flow when the product is discharged. In the subsequent fluid modules 12, only the valve 25 assigned to the supply media feeding channel 17 is closed. The product can thus flow through the complete fluid system 10.

Furthermore, the fluid system 10 can be brought into a cleaning state.

For cleaning, two fluid channels 16, 18 or valves 24 and 26 of a fluid module are opened, while the other two fluid channels 17, 19 or valves 25 and 27 are closed. A cleaning medium can then be supplied through one of the two unblocked fluid channels, in particular the cleaning media feeding channel 16, and flow from the cleaning media input 42 to the media output 40 of a fluid module 12, the areas of the fluid module 12 in contact with the medium being thus cleaned. In particular the central fluid space is flowed through.

The cleaning medium is then discharged through the further unblocked fluid channel, in particular the outlet channel 18.

Depending on the requirements, various cleaning media such as, for example, ultrapure steam, water for injection purposes or chemical cleaning agents can be used successively or alternatively.

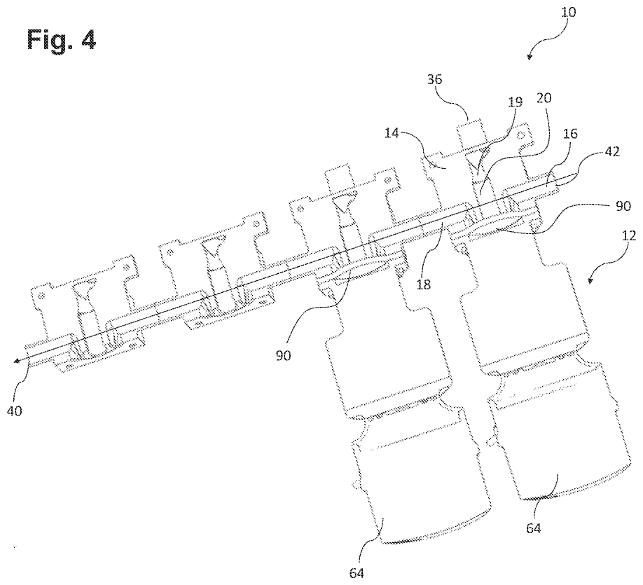

By respectively connecting the media output 40 of the fluid system 10 to the cleaning medium input 42 of a subsequent fluid module 12, the cleaning medium can flow through all fluid modules 12 of the fluid system 10 if the valves are switched accordingly. This cleaning medium flow is illustrated by an arrow in FIG. 4.

To improve the cleaning effect, at least one of the two unblocked fluid channels 16, 18 can be repeatedly closed and reopened in a pulse-like manner during cleaning. In other words, one or more valves 24, 26 may be switched in a clocked manner.

In addition to the fluid modules 12, the fluid system 10 further comprises an output module 44 arranged on an output side 46 of the fluid system 10.

The output module 44 has a media input 48, a media output 49, and a cleaning media output 50. Furthermore, the output module 44 comprises three individual valves 51, 52, 53, in particular an input valve 51, an output valve 52, and a cleaning media output valve 53.

A cleaning medium collecting container 54 is arranged at the cleaning medium output 50, which is fluidically connected to the cleaning medium output 50. The cleaning medium can be collected therein after the purging of the fluid system 10.

A product container 55 is arranged at the media output 49, which is fluidically connected to the media output 49 and in which a product removed from the containers 32 can be collected.

The containers 32 contain, for example, cell cultures which are repeatedly supplied with a supply medium or with different supply media during a growth phase over a longer period of time, for example over several weeks.

The containers 32 are usually uncoupled from the fluid system 10 after a supply with supply medium or after removal of a product from the containers 32, such that a fluid system 10 is not continuously occupied. In the meantime, additional containers 32 may be coupled to the fluid system 10.

At the end of the growth phase of the cell cultures, removal of the product usually occurs.

Due to the possibility of cleaning the fluid system 10 as previously described, the risk of contaminating a container 32 with residues of an incorrect supply medium is minimized.

For example, cleaning may be performed each time a container 32 is uncoupled from the fluid system 10 and/or each time a container 32 is coupled to the fluid system 10.

The coupling module 28 may be cleaned in an autoclave prior to assembly to the valve node 14.

FIGS. 2 to 4 show a fluid system 10 according to the invention having a plurality of, in particular four fluid modules 12, FIG. 4 showing a section through the fluid system 10. The output module 44 shown schematically in FIG. 1 is not shown for reasons of simplicity.

FIGS. 2 to 4 show the individual fluid modules 12 in different stages of assembly for better illustration, a fully assembled fluid module 12 being shown on the left in FIG. 2.

Each of the fluid modules 12 includes a valve node 14 having a base body 22 delimited by an outer surface 56. A plurality of connections 57 are provided on the base body 22 and are assigned to the fluid channels 16, 17, 18 in the base body 22.

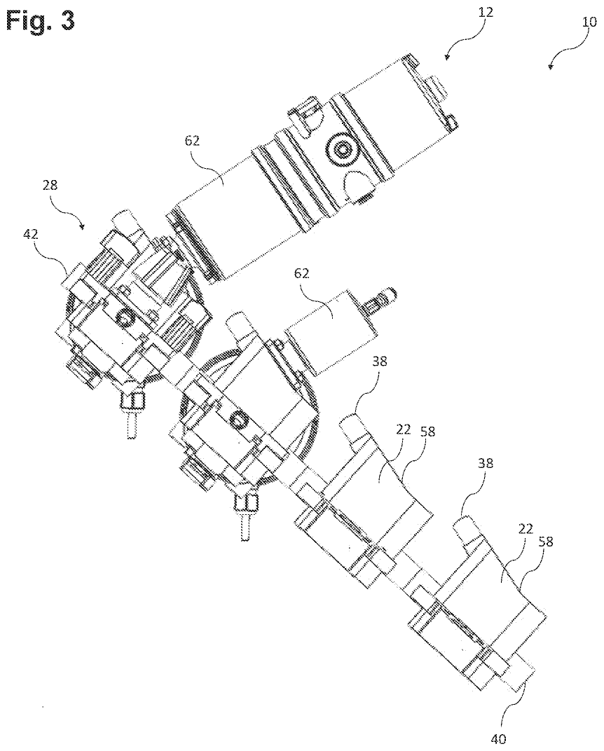

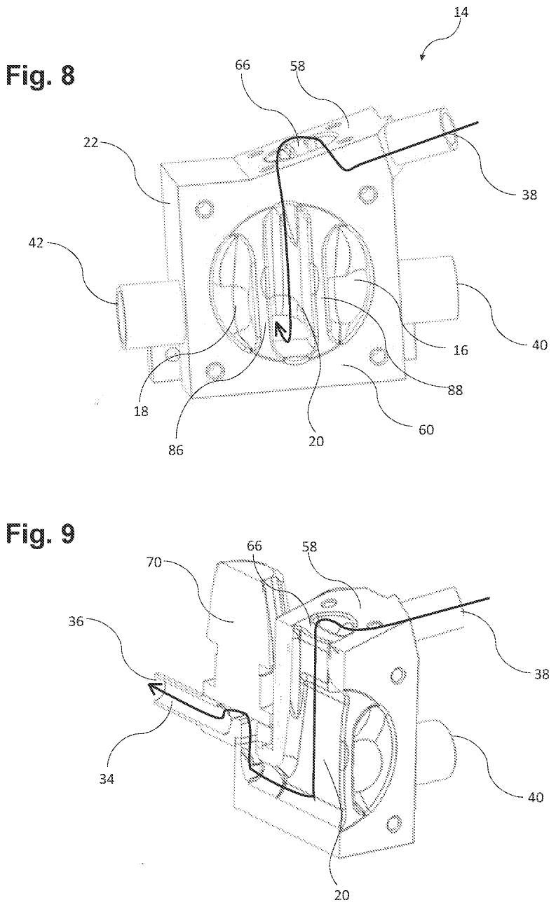

Two valve connections 58, 60 are provided on the outer surface 56, only the valve connection 58 being visible in FIGS. 2 and 3 and the further valve connection 60 (see FIG. 8) being concealed.

In the fully assembled state of a fluid module 12, a valve actuator 62, 64 is provided at each valve connection 58, 60 of the base body 22. In the example embodiment shown, the valve actuators 62, 64 are flanged to the valve node 14.

The valve actuator 62 assigned to the supply media feeding channel 17 is, for example, a two-way diaphragm valve actuator. Two different embodiments are shown for the valve actuator 62 in FIGS. 2 and 3, which can selectively be used in the fluid system 10.

The valve actuators 64 assigned to the cleaning media feeding channel 16 and the outlet channel 18, respectively, are each a so-called multiport valve actuator.

As can be seen in FIG. 2, a valve seat 66 is provided in the area of the valve connection 58. A closure body which can be moved by the valve actuator 62 can cooperate with the valve seat 66.

FIGS. 2 and 3 further illustrate the fastening of the coupling module 28 at the coupling point 30. In addition, it can be seen in FIG. 2 that the transfer channel 19 opens at the coupling point 30.

Each coupling module 28 comprises a coupling valve housing 68 having a valve seat 69. Further, the coupling module 28 comprises a closure body, which is not shown in the figures, and a valve actuator 70 for the closure body. The closure body of the coupling module 28 is for example a diaphragm.

The closure body cooperates with the valve seat 69 in the coupling valve housing 68. The illustrated valve actuator 70 can be operated by hand. However, it is also conceivable to use a valve actuator which can be driven hydraulically, pneumatically or electrically.

A seal 72, in particular an FDA molded seal, is arranged between the base body 22 and the coupling module 28. It surrounds a discharge opening 21 of the transfer channel 19.

FIG. 3 shows the fluid system 10 in a possible installation position. In this installation position, the fluid system 10 is arranged in an inclined manner such that there is a slope between the cleaning medium inlet 42 of the first fluid module 12 and the media output 40 of the last fluid module 12 of the fluid system 10. Due to this slope, the fluid system 10 is self-draining. Furthermore, this installation position enables a media transport without auxiliary energy. However, it is also conceivable to use a pump to support the media transport.

The installation angle or the slope that is necessary for a self-draining depends on the nominal width of the transfer channel 19 and the seat contour of the valve seat 69 of the coupling module 28. In order to mark the installation angle, there can be a marking on the fluid module 12, which must be at the top when the fluid module 12 is installed.

The described installation position also prevents the mixing of media in the event of a defect or malfunction of the multiport valve actuator and allows the internal volume to the coupling module 28 to be kept low.

Three of the total of four fluid channels 16, 17, 18, 19, which are fluidically separated from each other and are present in the base body 22, as well as the central fluid space 20 can be seen in the sectional view in FIG. 4. More precisely, the cleaning media feeding channel (fluid channel 16), the outlet channel (fluid channel 18) and the transfer channel (fluid channel 19) can be seen. Only the supply media feeding channel (fluid channel 17) cannot be seen in FIG. 4. All fluid channels 16, 17, 18, 19 open into the central fluid space 20.

When the coupling module 28 is fastened to the coupling point 30, the transfer channel 19 is fluidly connected to the connection channel 34 formed in the coupling module 28. Thus, the transfer channel 19 and the connection channel 34 together form a connecting channel from the central fluid space 20 to the connection 36.

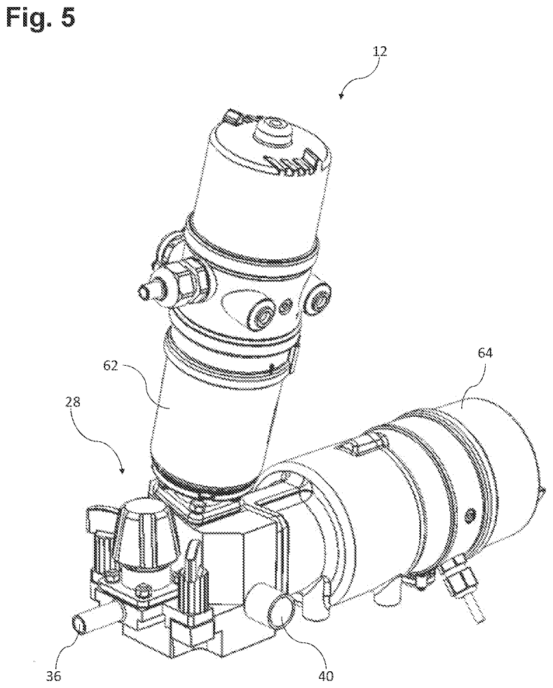

FIG. 5 shows a single, fully assembled fluid module 12.

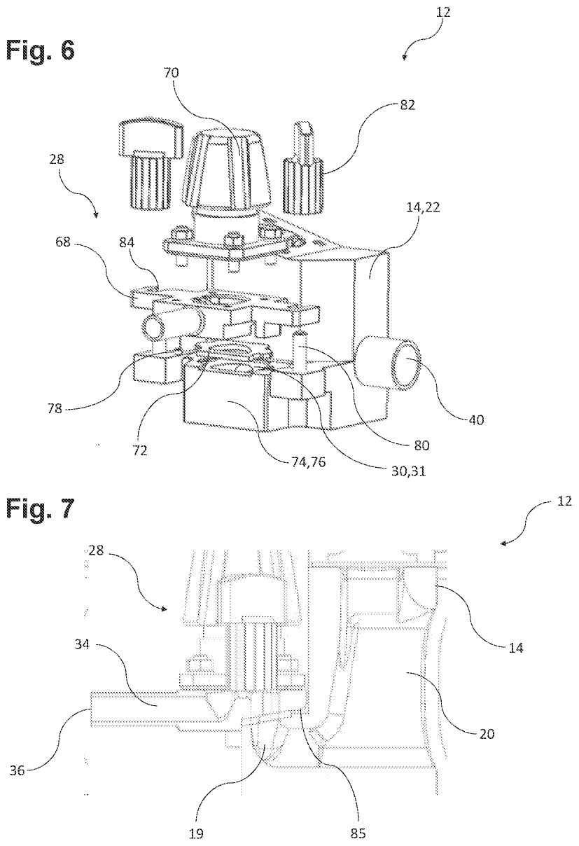

FIG. 6 shows an exploded view of a fluid module 12 having a valve node 14 and a coupling module 28.

As can already be seen in FIG. 2, the coupling point 30 is formed by a coupling flange 74, which is formed by a projection 76 of the base body 22. In particular, the base body 22 is L-shaped and the coupling point 30 is formed on an inner side of one of the legs of the L.

In particular, the coupling point 30 has a coupling face 31 against which the coupling module 28 rests.

In the embodiment shown in FIG. 6, the coupling face 31 extends in sections in an inclined manner relative to a flow direction of the fluid in the area of the coupling point 30, for example by an angle between 5° and 15°. The coupling face 31 in particular encloses an obtuse angle with an inner side of the further leg of the L in the area of the inclination, for example an angle between 95° and 110°. The inclination ensures that the seal 72 is not damaged when the coupling module 28 is fastened. Thus, it is not necessary for the entire coupling face 31 to be inclined, rather, it is sufficient if the part of the coupling face 31 in which the seal is arranged is inclined.

The coupling valve housing 68 has a correspondingly inclined surface, such that the coupling module 28 can be positioned in a substantially horizontal orientation.

It can also be seen in the exploded view that positioning means 78 and fastening means 80 are provided on the valve node 14.

The positioning means 78 are formed by elevated portions which can engage corresponding recesses in the coupling valve housing 68 in a force-fitting and/or form-fitting manner. The positioning means 78 can be spring-mounted so that they can be pressed into the base body 22 when pressure is applied and can latch in the coupling valve housing 68 when the end position of the coupling module 28 is reached.

In particular, the fastening means 80 are threaded bolts onto which a fixing element 82 can be screwed to fix the coupling module 28 to the valve node 14.

The fixing elements 82 may include a screw nut. In addition, the fixing elements 82 have wings that allow for one-handed operation without tools.

The coupling valve housing 68 has corresponding cavities 84, in which the fastening means 80 can engage. Preferably, the cavities 84 are open in one direction to facilitate assembly so that the coupling valve housing 68 can be easily pushed onto the valve node 14.

To prevent damage to the seal 72 when pushing the coupling module 28 on, the coupling module 28 is rounded at an edge 85 facing the valve node 14 (see FIG. 7).

By detaching the fastening means 80, the coupling module 28 can be detached from the valve node 14 in a non-destructive manner, if required.

FIG. 7 shows a partial section through the valve node 14 shown in FIG. 6 with the coupling module 28 fastened thereto. FIG. 7 shows the course of the connecting channel formed by the transfer channel 19 and the connection channel 34.

FIG. 8 shows a perspective view of the valve node 14, in which the valve connections 58 and 60 can be seen.

FIG. 8 further shows that the central fluid space 20 is openly accessible from at least one side of the base body 22 and, in a plan view of said side, a respective web 86, 88 is formed in the base body 22 on either side of the central fluid space 20, each of the two webs 86, 88 forming a valve seat.

Furthermore, each of the two webs 86, 88 delimits the central fluid space 20 with respect to a respective one of the fluid channels 16, 18 adjacent thereto, in particular with respect to the cleaning media feeding channel (fluid channel 16) and the outlet channel (fluid channel 18).

The fact that the webs 86, 88, which each form a valve seat, are accessible from the same side makes it possible to use the multiport valve actuator 64 mentioned above. This means that a common valve actuator 64 is assigned to two individual valves, in the present case the valve 24, which is assigned to the cleaning media feeding channel 16, and the valve 26, which is assigned to the outlet channel 18. The two valves 24, 26 can be controlled simultaneously or independently of each other.

A common closure body 90 (see FIG. 4) is assigned to the two webs 86, 88 for this purpose. The closure body 90 is a diaphragm which can be selectively pressed in a sealing manner against each of the webs 86, 88 by the valve actuator 64.

FIG. 9 shows a perspective sectional view of the valve node 14 shown in FIG. 8 with a coupling module 28 fastened thereto. The valve actuator 70 is shown in FIG. 9 in a highly simplified form without an inner workings.

In FIGS. 8 and 9, an arrow is used to illustrate a supply media flow. In particular, the supply media flow flows from the supply media input 38 via the valve seat 66 into the central fluid space 20, and from there through the transfer channel 19 into the connection channel 34 of the coupling module 28.

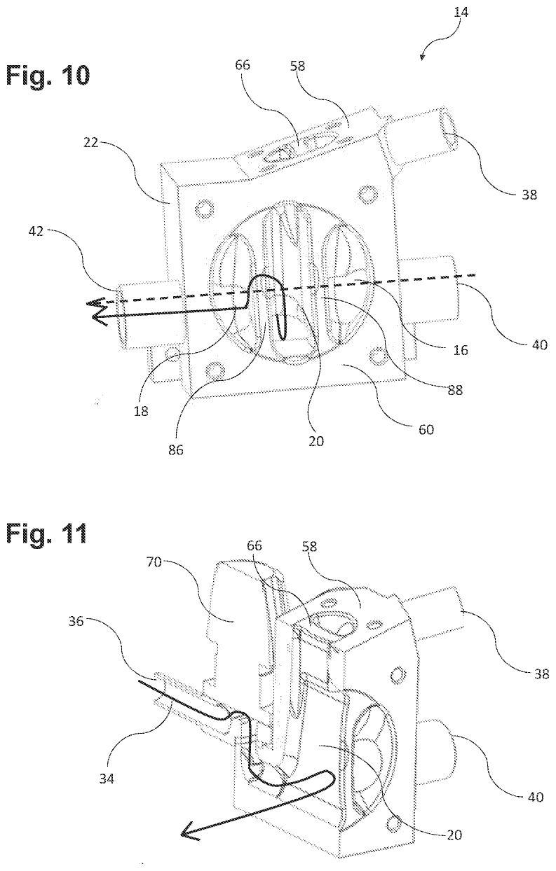

FIGS. 10 and 11 also show a perspective view of a valve node 14 and a sectional view through a valve node 14 with a coupling module 28 fastened thereto, respectively.

In FIGS. 10 and 11, an arrow is used to illustrate a product flow instead of a supply media flow. The product flow passes from the connection 36 through the connection channel 34 in the coupling module 28 and the transfer channel into the central fluid space 20, and from there to media output 40.

FIG. 10 additionally illustrates the cleaning media flow with a dashed arrow.

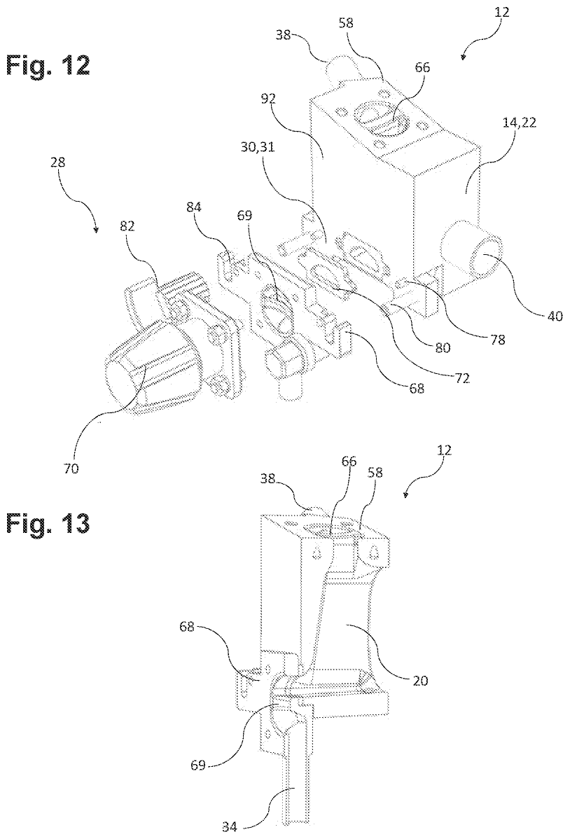

FIGS. 12 and 13 show a valve node 14 with a coupling module 28 according to a further embodiment in an exploded view and in a perspective sectional view.

For identical structures having identical functions, which are known from the previously described embodiment, the same reference numerals are used in the following, and in this respect, reference is made to the previous explanations, the differences with respect to the previous embodiment being discussed below in order to avoid repetition.

The embodiment illustrated in FIGS. 12 and 13 differs from the previously described embodiment in the spatial orientation of the coupling point 30 and the coupling face 31, respectively.

Here, the base body 22 is not L-shaped, but is essentially parallelepiped-shaped. More precisely, the base body 22 has the shape of a parallelepiped having an attached ramp.

The coupling point 30 or the coupling face 31 is provided on a side face 92 of the parallelepiped.

In addition, the fluid module 12 illustrated in FIGS. 12 and 13 differs from the previously described embodiment in the orientation of the coupling module 28 relative to the valve node 14, mainly due to the different orientation of the coupling face 31. The coupling module 28 is oriented substantially vertically.

In the installed position, the connection channel 34 formed in the coupling module 28 extends downward, whereas in the previously described embodiment, the connection channel 34 extends substantially horizontally in the installed position.

The embodiment illustrated in FIGS. 12 and 13 has the advantage that a fluid can flow particularly quickly through the coupling module 28 to a container 32 due to the orientation of the connection channel 34.

The previously described embodiment, on the other hand, is equally well suited for supplying a fluid to and removing a fluid from a container 32, respectively.

1. A fluid module having a valve node which includes a base body delimited by an outer surface and having a central fluid space, and at least four fluid channels which are fluidically separated from each other and each open into the central fluid space, wherein at least two valve connections are provided on the outer surface, and wherein a coupling point is provided on the valve node, a coupling module being fastened to the coupling point, a connection channel being formed in the coupling module, the connection channel being fluidically connected to one of the fluid channels in the valve node, and wherein the coupling module comprises a coupling valve which selectively unblocks and closes the connection channel.

2. The fluid module according to claim 1, characterized in that the coupling module is non-destructively detachably fastened to the valve node.

3. The fluid module according to claim 1, characterized in that the coupling module includes a coupling valve housing having a valve seat, a closure body cooperating with the valve seat, and a valve actuator for the closure body.

4. The fluid module according to claim 1, characterized in that a seal is arranged at the coupling point between the valve node and the coupling module, which surrounds a discharge opening of the fluid channel opening into the coupling point.

5. The fluid module according to claim 1, characterized in that the coupling point is formed by a coupling flange which is formed by a projection of the base body

6. The fluid module according to claim 5, wherein the base body is L-shaped and the coupling point is present on an inner side of one of the legs of the L.

7. The fluid module according to claim 5, characterized in that the coupling point has a coupling face against which the coupling module rests, the coupling face extending in an inclined manner relative to a flow direction of a fluid in the area of the coupling point.

8. The fluid module according to claim 1, characterized in that the base body is substantially parallelepiped-shaped and the coupling point is provided on a side face of the parallelepiped.

9. The fluid module according to claim 1, characterized in that a valve actuator is assigned to each valve connection of the base body.

10. The fluid module according to claim 1, characterized in that positioning means and/or fastening means are provided on the valve node and/or on the coupling module to fasten the coupling module in a defined position on the valve node.

11. The fluid module according to claim 1, characterized in that positioning means and/or fastening means are provided on the coupling valve housing to fasten the coupling module in a defined position on the valve node.

12. The fluid module according to claim 1, characterized in that the central fluid space is openly accessible from at least one side of the base body and, in a plan view of said side, a respective web is formed in the base body on both sides of the central fluid space, each of the two webs forming a valve seat and delimiting the central fluid space with respect to a respective one of the fluid channels adjacent thereto.

13. The fluid module according to claim 12, characterized in that a common closure body is assigned to the two webs, wherein the closure body is a diaphragm which can selectively be pressed in a sealing manner against each of the webs by a valve actuator.

14. A fluid system comprising at least two fluid modules,
each fluid module having a valve node which includes a base body delimited by an outer surface and having a central fluid space, and at least four fluid channels which are fluidically separated from each other and each open into the central fluid space, wherein at least two valve connections are provided on the outer surface, and wherein a coupling point is provided on the valve node, a coupling module being fastened to the coupling point, a connection channel being formed in the coupling module, the connection channel being fluidically connected to one of the fluid channels in the valve node, and wherein the coupling module comprises a coupling valve which selectively unblocks and closes the connection channel,
wherein the individual fluid modules are connected to each other such that an output fluid channel of the fluid channels of one fluid module is fluidically connected to an input fluid channel of the fluid channels of the at least one further fluid module.

15. A method of cleaning a fluid system according to claim 14, comprising the following steps:
closing two of the four fluid channels of one of the at least two fluid modules and unblocking the two remaining fluid channels,
supplying a cleaning medium through one of the two unblocked fluid channels, wherein the central fluid space is flowed through, and
discharging the cleaning medium through the further unblocked fluid channel.

16. The method according to claim 15, characterized in that during cleaning, at least one of the two unblocked fluid channels is repeatedly closed and reopened in a pulse-like manner.

17. A method of cleaning a fluid module according to claim 1, comprising the following steps:
closing two of the four fluid channels of the fluid module and unblocking the two remaining fluid channels,
supplying a cleaning medium through one of the two unblocked fluid channels, wherein the central fluid space is flowed through, and
discharging the cleaning medium through the further unblocked fluid channel.

18. The method according to claim 17, characterized in that during cleaning, at least one of the two unblocked fluid channels is repeatedly closed and reopened in a pulse-like manner.
